(19) United States
(12) Patent Application Publication
(10) Pub. No.: US 2015/0359660 A1
(43) Pub. Date: Dec. 17, 2015

(54) HYDRO-BLOCK AIR VENT FEMALE EXTERNAL CATHETER AND METHOD OF USE

(71) Applicant: Mark Harvie, Williston, VT (US)

(72) Inventor: Mark Harvie, Williston, VT (US)

(21) Appl. No.: 14/835,759

(22) Filed: Aug. 26, 2015

Related U.S. Application Data
(63) Continuation of application No. 14/794,787, filed on Jul. 8, 2015.
Application No. 14/794,787 is a continuation of application No. 14/270,467, filed on May 6, 2014.
(60) Provisional application No. 62/041,894, filed on Aug. 26, 2014.

(51) Int. Cl. A61F 5/455; A61F 5/44; A61F 5/441

(52) U.S. Cl. CPC A61F 5/455; A61F 5/441; A61F 5/4404; A61F 5/443

(57) ABSTRACT

This invention is a device that utilizes hydro-block venting material in various external female catheter configurations to permit adequate airflow into the catheter thereby permitting efficient and complete drainage of urine away from the user following urination. In one embodiment the user's expelled urine is drawn away from the user by a pump means to a user selected storage means. In another embodiment gravity is utilized to draw the user's expelled urine away from the user to a user selected storage means. In another embodiment a comfort ring is attached to the device thereby reducing irritation and discomfort when the device is in place during use. An adhesive, garment or strap attachment means are disclosed as attachment means to secure the external female catheter to the user and prevent leakage.

### CROSS REFERENCE TO RELATED APPLICATION

This application is a Continuation-In-Part of the Co-Pending patent application U.S. Ser. No. 14/794,787, filed Jul. 8, 2015. This application is also a Continuation-In-Part of the Co-Pending patent application U.S. Ser. No. 14/270,467, filed May 6, 2014, which claimed priority from Provisional Patent Application No. 61/819,833 which was filed on May 6, 2013. This application also claims priority from the Provisional Patent Application No. 62/041,894 which was filed on Aug. 26, 2014.

### BACKGROUND ART

The English word “catheter” comes from the Greek word kathienai, meaning “to let or send down.” The historical and archaeological record teaches that catheters were used as early as 3,000 B.C.E. principally to relieve the pain of urinary retention. Ancient doctors used many different materials to form a hollow catheter shape which included: reeds of straw, rolled up palm leaves, the hollow tops of onions, gold, silver, copper, brass, and yes, even lead. The 11th century C.E. saw the advent of the malleable catheter. Eventually in later centuries silver was in more common use principally because it could be bent to a desired shape and was believed to have an antiseptic function. The antimicrobial properties of silver have been known to cultures all around the world for many centuries. The Phonecians stored water and other liquids in silver coated bottles to discourage contamination by microbes. In fact Benjamin Franklin invented a silver catheter for use by his brother who suffered from kidney stones and who needed to undergo the daily placement of his brother's bulky silver catheter into his bladder. To make this less painful Franklin designed a flexible catheter, albeit still of silver. Franklin is quoted as saying about his catheter that “[i]t is as flexible as would be expected in a thing of the kind, and I imagine will readily comply with the turns of the passage.”

The invention of rubber vulcanization by Goodyear® in 1844 not only improved the firmness of the catheter it dramatically increased the durability as well. The vulcanized rubber Coudé tip catheter was developed and in common use in the late 18th and early 19th centuries principally to provide for male catheterization and these types of male catheters continue to be used in current medical practice today. Because the catheters during this time period were made from rubber they proved to be very weak at body temperature and caused serious health concerns. The catheter would break down and leave debris in the bladder that led to serious complications, not the least of which was bladder infections. Another major problem for these indwelling catheters was holding them in place. The earliest self retaining catheters had Malecot wing tips or Pezzer flexible shoulders that were tied to the male penis or sutured to the female labia to maintain the catheter in a functional position.

The invention of latex rubber and that of neoprene by Dupont™ in the early 1930s solved many significant problems that were being experienced with the vulcanized rubber indwelling catheters. The first artificial sphincter designed as an inflatable circular cuff was created by Frederic Foley in 1947. Foley is best known for the improvement of the principles of the transurethral indwelling balloon catheter and he played a major role in the introduction of commercially manufactured indwelling balloon catheters in the 1930s. The Foley catheter is widely used in multiple health care settings to this day.

The concept of sterile intermittent catheterization in patients with spinal cord injury was first introduced after World War II. Dr. Jack Lapides while at the University of Michigan at Ann Arbor introduced the clean intermittent catheterization technique in 1971. Despite the fact that sterile technique had been used for catheterization for many years, urinary tract and bladder infections were frequent occurrences among patients that had been catheterized. Lapides concluded that bacteria were not the only cause of infections among these patients. He first posited the accepted conclusion that chronic stagnant urine residuals and overstretching of the bladder are also responsible for these infections. Although Lapides was criticized in the medical community regarding his conclusions he held fast to his conclusion that sterile intermittent catheterization was superior to indwelling catheters. More than four decades later the clean intermittent catheterization recommended by Lapides remains the preferred method to treat chronic urine retention and a neurogenic bladder.

The indwelling or intermittent catheterization of the bladder has historically been viewed as safe because of the antiseptic methods of use and manufacture that have become common practice. From the beginning up to today, however, many physicians are very concerned about catheter related infections. Countless patients are still developing systemic infections (catheter fever) despite the use of antiseptic methods of manufacture and use.

It has become fairly standard medical protocol for the treatment of women to keep internal catheters placed for as long as they remain unable to use a bathroom facility. This sadly results in frequent and severe bladder or urinary track infections. These hospital-acquired infection (HAI) or nosocomial infections are infections whose developments are favored by a hospital environment. These nosocomial infections have been determined to affect approximately five percent of hospitalized women. These infections are very painful and can often lead to disability, longer hospital stays and even death. Worldwide the treatment of indwelling catheter generated nosocomial infections costs the healthcare system billions of dollars each year. In fact, indwelling catheter generated hospital-acquired infections account for as much as forty percent of all nosocomial infections.

The medical community appears to have concluded that the incidents of indwelling catheter generated nosocomial infections can be reduced, if not eliminated, with an effective external catheter being used in place of an indwelling device. The problem for the medical community is that very few such female external collection devices have been developed in the art and none of them afford an effective solution to this chronic problem.

In view of these problems there have been a number of attempts in the prior art female external catheters to solve these problems. The prior art teaching these devices appear in U.S. Pat. No. 3,601,125 to Moss; U.S. Pat. No. 4,233,978 to Hickey; U.S. Pat. No. 4,563,183 to Barrodale; and U.S. Pat. No. 4,615,692 to Giacalone. Some of the prior art teach a device that has a projection adapted for insertion into the vagina itself such as found in U.S. Pat. No. 3,776,235 to Ratcliffe; U.S. Pat. No. 4,194,308 to Anderson; U.S. Pat. No. 4,198,979 to Cooney; said U.S. Pat. No. 4,563,183; and said U.S. Pat. No. 4,615,692. U.S. Pat. No. 3,349,768 to Keane also attempts to solve these problems with a portable urinal with a suction means designed to fit a female patient. The interface with the female genitalia has a suction head designed to receive a removable pad of open celled porous material.

Additionally, U.S. Pat. No. 4,202,058 teaches a female urinal that utilizes a lined receptacle with an attached drainage tube that can be connected to a pump and the tube is comprised of of a corrugated or cellulose portion with one way valves at each end to provide suction for removing urine from the urinal portion of the device. U.S. Pat. No. 4,246,901 teaches a urine collection device that is funnel shaped and filled with a wicking material to soak up discharged urine.

A multiple layer absorbent pad is taught in U.S. Pat. No. 4,360,015. Two layers of absorbent material in this device are separated by a grid material and are covered on one side by a moisture permeable layer and on the other side by an impermeable layer. This absorbent methodology is also taught in U.S. Pat. No. 4,798,603 where the absorbent material is made up of a substantially hydrophilic material with a facing and a liquid permeable top sheet layer of hydrophobic material. The device also has a liquid permeable transport layer placed between the top sheet and the absorbent material that is made of a material less hydrophobic than the patient's body.

U.S. Pat. No. 4,747,166 is a liquid aspiration system that includes an absorptive pad with an inner core of urine absorbing material, an upper pad facing layer of liquid permeable hydrophobic material in contact with the user and a lower pad backing of impermeable material. The pad has a flexible perforated tube with a liquid outlet connector at one end. The device has a vacuum or pump, a urine collection means and vacuum tubing coupling the tube within the pad to the collection means and the vacuum or pump. The vacuum or pump may run continuously or be activated by a sensor in the pad.

U.S. Pat. No. 4,631,061, is an automatic urine detecting, collecting and storing device with a urine collection means that has sensors for detecting the presence of urine which then activates a pump to draw urine from the collection means to a storage means.

U.S. Pat. No. 4,610,675, is a female urine collection device that is a flexible pad with an elongated central opening for partial vaginal insertion within the external area of the genitalia. The absorbent core has a plurality of layers of material.

U.S. Pat. No. 4,981,474, teaches a urine drainage device that has a wedge shaped reservoir with hinged sidewalls that are joined along a common edge with a collapsible sidewall. Spring inside the device hold the hinged sidewalls apart and inlet and outlet ports with a one way valve to facilitate the flow of urine.

U.S. Pat. No. 4,886,508 teaches an external catheter assembly for women which has a panty support for proper placement of the collection means. The panty and the collection means cooperate such that the urine collection means' position can be adjusted relative to the panty to optimize comfort and function.

It is known in the art that most complications that occur with external catheters occur when the catheter is either used incorrectly or used for a longer period of time than intended. The most common complications that can occur are:
Skin irritation or urinary tract or bladder infections.
Constant wet skin can become soft and worn away by the external catheter.
Allergic reactions to the materials can develop as well.

All of the prior art above does not effectively permit urine to escape the initial collection area thus leaving a user wet with their own urine. This is so even when a vacuum or pump means is utilized to draw urine away from the user because the materials used in the primary collection area adjacent to the urethra do not provide an adequate venting means to permit the urine to completely evacuate and the area to dry.

Thus, while there are a number of prior art attempts to solve the problem of patients remaining wet while wearing these devices and developing infections, etc., none of these devices are satisfactory solutions. Therefore there is a great need for an external female catheter device that is easily donned and remains securely in place regardless of the activity level of the user. Furthermore, to solve the problems of the prior art devices this external female catheter must also effectively facilitate the aspiration of backwashed urine that may accumulate between the genitalia, labium and perineum and the external female catheter that is unable to drain from the distal or discharge end of the catheter. My invention is designed to solve these problems that exist in the current art.

### DISCLOSURE OF THE INVENTION

### Purpose of Invention

The purpose of the invention is to provide an external female catheter that will permit air to enter into the device by means of a Hydro-block or similar substance air vent that allows air to flow into the external female catheter while blocking the urine from exiting except through the distal end drain as intended. This invention will allow the urine to completely drain out of the external female catheter and the hose and completely away from the user's skin.

### Description of Problems Solved by Invention

There are currently no external female catheter devices that will fit a user securely enough to allow for reasonable physical activity of a user that will also permit air to enter into the external female catheter and thereby allow any backwashed urine to escape, thereby preventing such adverse medical conditions as skin irritation, maceration of the genitalia, urinary tract infections (UTI), ischemia and vaginal edema or urethral obstruction. The current invention incorporates a proprietary Hydro-block air vent that allows air to flow into the external female catheter while blocking the urine from exiting except through the distal end intended drain. This invention will allow the urine to completely drain out of the external female catheter and any attached hose and ultimately away from the user's skin, unlike current art external female catheters which permit urine to accumulate and remain in contact with the user's skin and urethra.

My external female catheter invention incorporates a proprietary Hydro-block air vent that allows air to flow into the catheter while blocking the urine from exiting. This invention will allow the catheter to be very small in size, only a few millimeters deep and wide, allowing for a unique and multiple sizing for each female anatomy. The small size makes it possible for females to wear the system under normal clothing while hydro block air filters allow the catheter to drain completely under gravity in most positions. Also with the addition of my patented bladder relief sensor activated pump (U.S. Pat. No. 7,866,942), the urine is completely drained out of the external catheter away from the user's skin leaving them dry at all times no matter what physical orientation their body is in, (i.e. standing, walking, climbing, sitting, reclined, lying flat on back, raised legs or in a hospital bed, lying on either side, lying on stomach in fact even upside down). This will result in significant reduction in skin ulcers, maceration and UTI which are currently all major problems and are a significant cost to the healthcare system for inpatient hospital care, nursing home long term care and most importantly home health care resulting in significant reduction in complications and wound care caused by urinary incontinence in females.

The most critical function of the invention disclosed in these letters patent is the aeration of the external female catheter. There are a number of attachment means in the art for external female catheters. Therefore it would be obvious to one skilled in the art for example to use an adhesive, garment or strap types of attachment means to hold my invention with the Hydro-block or similar substance air vent in place.

### Brief Description of Invention

My invention is an external female catheter which incorporates a proprietary Hydro-block or similar substance air vent that allows air to flow into the catheter while blocking the urine from exiting except through the intended drainage site at the distal end of the catheter. This invention will allow the urine to completely drain out of the catheter and any attached hose and ultimately away from the user's skin.

### BRIEF DESCRIPTION OF THE DRAWINGS

FIG. 1 is an exploded perspective side view of the pump draining embodiment of the Hydro-Block Air Vent Female External Catheter.

FIG. 2 is a perspective side view of the gravity draining embodiment of the Hydro-Block Air Vent Female External Catheter without the Quick Disconnect Coupling and Drainage Hose attached.

FIG. 3 is a perspective side view of the gravity draining embodiment of the Hydro-Block Air Vent Female External Catheter without the Hydro-block Air Intake Coupler, Quick Disconnect Coupling and Drainage Hose attached.

FIG. 4 is a right side perspective top view of the pump draining embodiment of the Hydro-Block Air Vent Female External Catheter depicting the urine collection area of the Micro Collection Chamber.

FIG. 5 is an exploded perspective side view of the gravity draining embodiment of the Hydro-Block Air Vent Female External Catheter.

FIG. 6A is a left side perspective bottom view of the comfort ring embodiment of the Hydro-Block Air Vent Female External Catheter depicting the comfort ring attached.

FIG. 6B is a left side perspective bottom view of the comfort ring embodiment of the Hydro-Block Air Vent Female External Catheter depicting the comfort ring detached.

### BEST MODES FOR CARRYING OUT THE INVENTION

### I. Preferred Embodiments

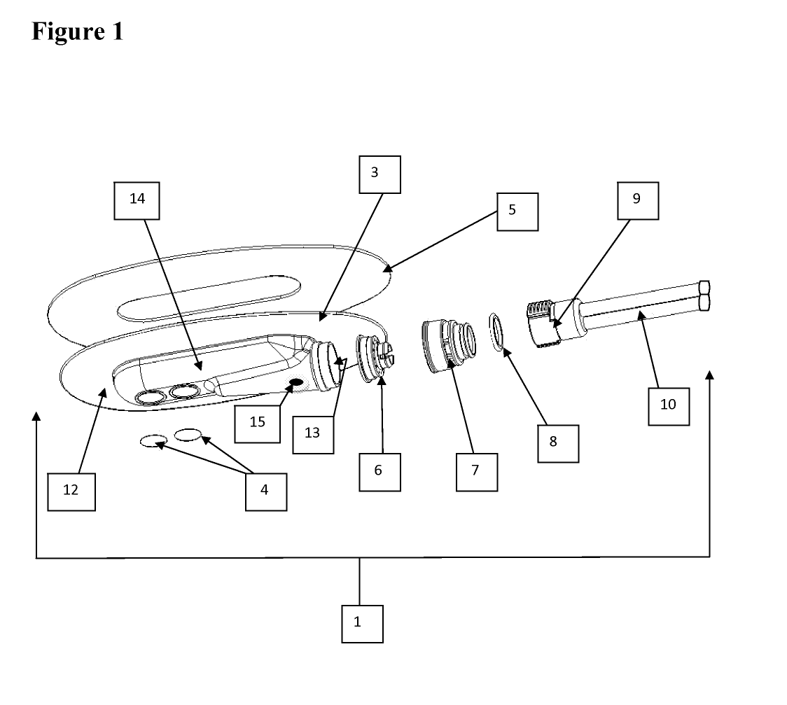

With reference now to the drawings, and in particular to FIGS. 1-6B thereof, are depicted three embodiments of a new and novel external female catheter utilizing hydro-block technology.

FIG. 1 depicts the Pump Draining embodiment of the Hydro-Block Air Vent External Female Catheter (1) which has at least one Urine Sensor (15) disposed in the Micro Collection Chamber (14) which is in electronic communication with a pump means to draw expelled urine from the Hydro-Block Air Vent External Female Catheter (1) to a user selected storage or disposal means. The Pump Draining embodiment of the Hydro-Block Air Vent External Female Catheter (1) is further comprised of Hydro-Block Air Intake Vents (4).

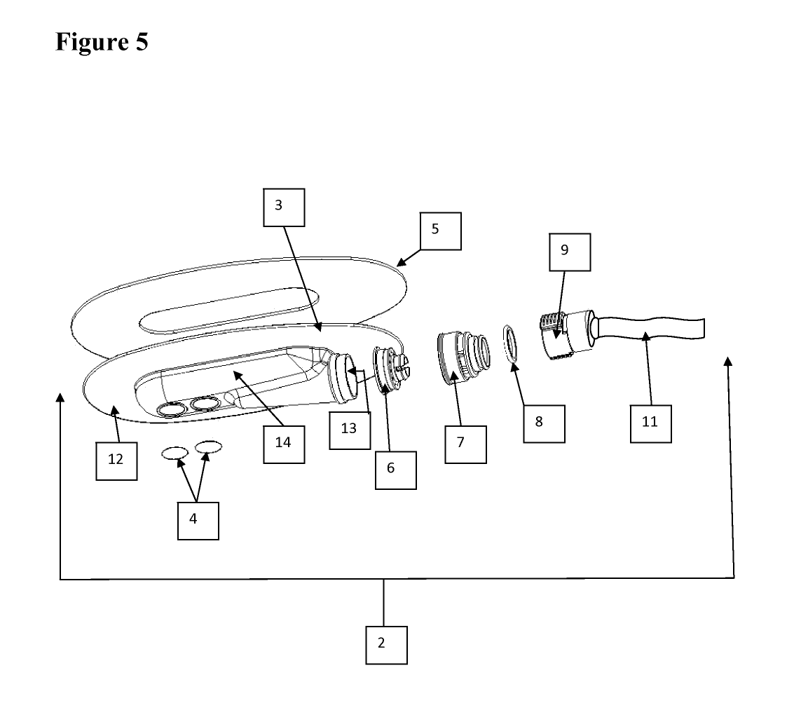

FIG. 5 depicts the Gravity Draining embodiment of the Hydro-Block Air Vent External Female Catheter (2) which utilizes gravity to draw expelled urine from the Hydro-Block Air Vent External Female Catheter (2) to a user selected storage or disposal means. The Gravity Draining embodiment of the Hydro-Block Air Vent External Female Catheter (2) is further comprised of Hydro-Block Air Intake Vents (4).

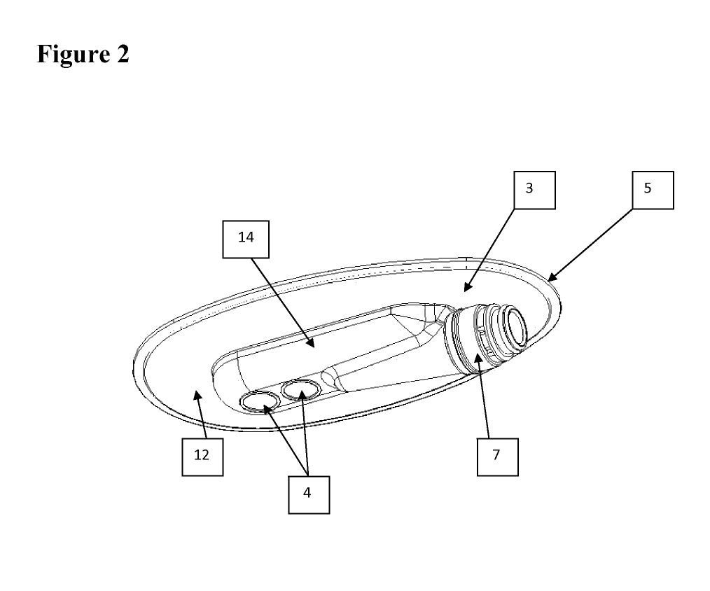

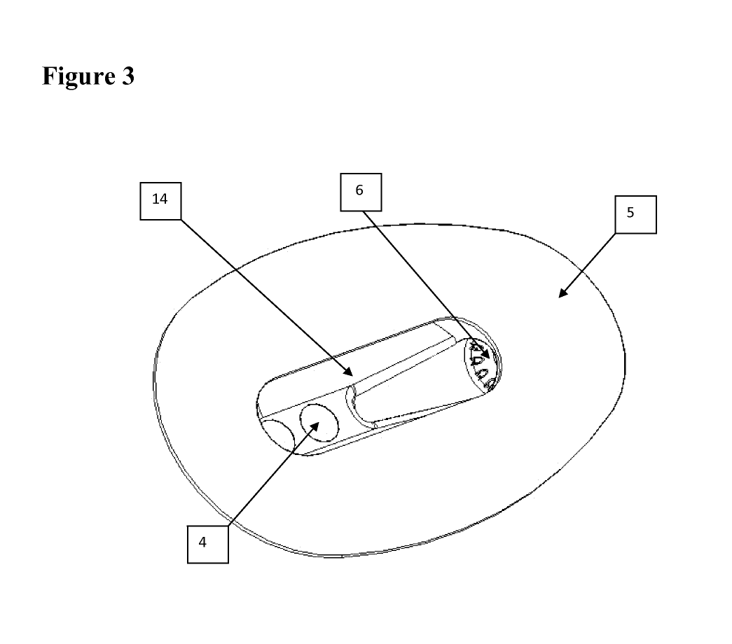

FIGS. 2 and 3 depict the Gravity Draining embodiment of the Hydro-Block Air Vent External Female Catheter (2) together with the Hydro-Block Air Intake Vents (4), but without the device's drainage components connected to the device.

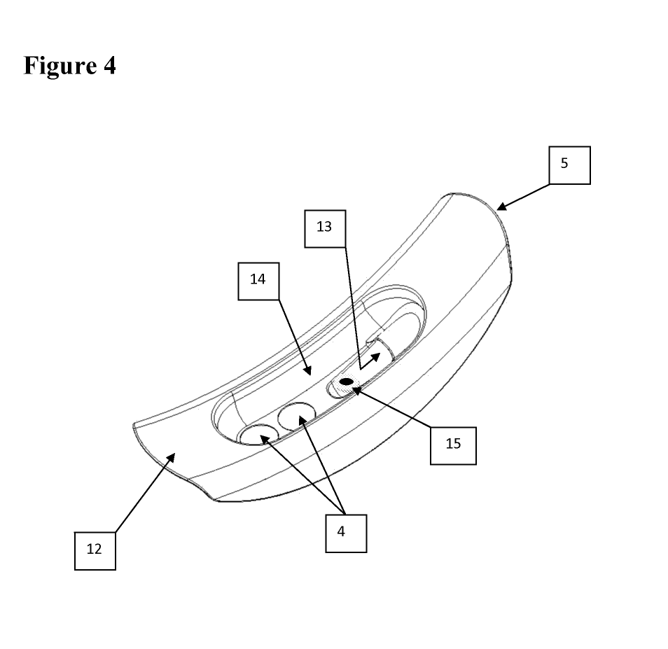

FIG. 4 depicts the Pump Draining embodiment of the Hydro-Block Air Vent External Female Catheter (1) with the Urine Sensor (15) disposed in the Micro Collection Chamber (14) together with the Hydro-Block Air Intake Vents (4), but without the device's drainage components connected to the device.

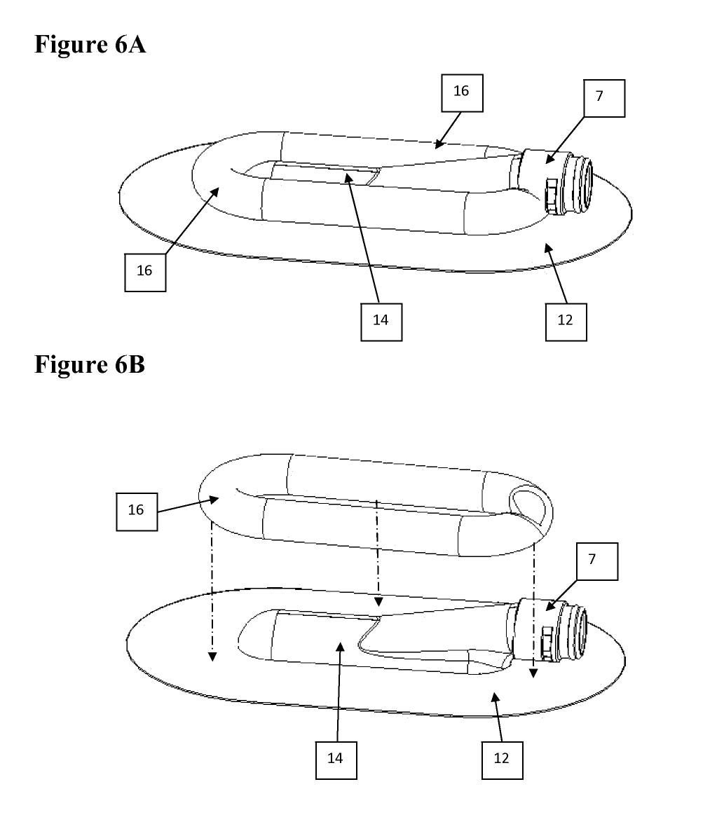

FIGS. 6A and 6B depict the Comfort Ring embodiment that may be used in both the Pump Draining embodiment of the Hydro-Block Air Vent External Female Catheter (1) and the Gravity Draining embodiment of the Hydro-Block Air Vent External Female Catheter (2) depicted in FIGS. 1-5.

### GENERAL DESCRIPTION OF REFERENCE NUMERALS IN THE DESCRIPTION AND DRAWINGS

Any actual dimensions listed are those of the preferred embodiment. Actual dimensions or exact hardware details and means may vary in a final product or most preferred embodiment and should be considered means for so as not to narrow the claims of the patent.

### LIST AND DESCRIPTION OF COMPONENT PARTS OF THE INVENTION

(1) Pump Draining embodiment of the Hydro-Block Air Vent External Female Catheter
(2) Gravity Draining embodiment of the Hydro-Block Air Vent External Female Catheter
(3) Body Contouring Sealing Sheet
(4) Hydro-block Air Intake Vent
(5) Hydrocolloid Adhesive
(6) Connector to Catheter Body Retainer
(7) Hydro-block Air Intake Coupler
(8) Interconnector Sealing O-Ring
(9) Quick Disconnect Coupling
(10) Pump Draining Drainage Hose
(11) Gravity Draining Drainage Hose
(12) External Catheter Molded Silicon Body
(13) Urine Passage Channel
(14) Micro Collection Chamber
(15) Urine Sensor
(16) Comfort Ring

### DETAILED DESCRIPTION OF THE PREFERRED EMBODIMENTS

1) The Pump Draining Embodiment of the Hydro-Block Air Vent External Female Catheter.

The Pump Draining embodiment of the Hydro-Block Air Vent External Female Catheter (1) depicted in FIGS. 1, 4, 6A and 6B is manufactured and comprised of the following components in their respective functional relationships:

The Pump Draining embodiment of the Hydro-Block Air Vent External Female Catheter (1) is formed in a size and shape capable of fitting between the legs and over the genitalia of a human female and forming a generally fluid tight seal around the vagina against the perineum, the mons pubis and the labia majora by securing the device thereto with a user selected attachment means, such as an adhesive means, garment or straps to complete the seal. In the Pump Draining embodiment of the Hydro-Block Air Vent External Female Catheter (1) the device is held in place by the Hydrocolloid Adhesive (5) which the user places against her perineum, mons pubis and labia majora to form a relatively fluid tight seal thereby such that expelled urine will be captured by the device. The Hydrocolloid Adhesive (5) is attached to the Body Contouring Sealing Sheet (3) which in turn is attached to the External Catheter Molded Silicon Body (12) which is molded to form a catch basin or urinal Micro Collection Chamber (14). The Micro Collection Chamber (14) has a portion molded to form a hole there through designed to accept and hold in place by a fluid tight seal at least one Hydro-block Air Intake Vent (4). The Micro Collection Chamber (14) has attached thereto at least one Urine Sensor (15). Upon urination the Urine Sensor (15) detects the presence of the urine and by electronic means (radio, WiFi, direct cabling) the presence of urine is communicated to a pump or vacuum means which is then activated creating a suction within the Micro Collection Chamber (14) through the Pump Draining Drainage Hose (10) which is connected to the Micro Collection Chamber (14) by means of the Connector to Catheter Body Retainer (6) which is attached to a Hydro-block Air Intake Coupler (7) that maintains a fluid tight seal by means of a Interconnector Sealing O-Ring (8) to the Quick Disconnect Coupling (9) that is attached to the Pump Draining Drainage Hose (10).

Once a user has placed the device over her genitalia and it is attached to the user as heretofore described the user may pass urine into the device. During the process of urination gravity and excretion pressure from the user's bladder and urethra will cause the urine to flow into the Micro Collection Chamber (12) and then be drawn away from the genitalia by the activated pump or vacuum means through the Urine Passage Channel (13) and away from the user into a user selected collection or disposal means. The urine will not be able to accumulate around the genitalia because the Hydro-block Air Intake Vent (4) and the Hydro-block Air Intake Coupler (7) will provide free air flow into the device thereby causing the urine to completely drain away from the user's genitalia and ultimately discharge through the Pump Draining Drainage Hose (10).

The Pump Draining embodiment of the Hydro-Block Air Vent External Female Catheter (1) may be manufactured from any suitably non-allergenic flexible material such as silicone rubber to optimize comfort to the user.

2) The Gravity Draining Embodiment of the Hydro-Block Air Vent External Female Catheter.

The Gravity Draining embodiment of the Hydro-Block Air Vent External Female Catheter (2) depicted in FIGS. 2, 3, 5, 6A and 6B is manufactured and comprised of the following components in their respective functional relationships:

The Gravity Draining embodiment of the Hydro-Block Air Vent External Female Catheter (2) is formed in a size and shape capable of fitting between the legs and over the genitalia of a human female and forming a generally fluid tight seal around the vagina against the perineum, the mons pubis and the labia majora by securing the device thereto with a user selected attachment means, such as an adhesive means, garment or straps to complete the seal. In the Gravity Draining embodiment of the Hydro-Block Air Vent External Female Catheter (2) the device is held in place by the Hydrocolloid Adhesive (5) which the user places against her perineum, mons pubis and labia majora to form a relatively fluid tight seal thereby such that expelled urine will be captured by the device. The Hydrocolloid Adhesive (5) is attached to the Body Contouring Sealing Sheet (3) which in turn is attached to the External Catheter Molded Silicon Body (12) which is molded to form a catch basin or urinal Micro Collection Chamber (14). The Micro Collection Chamber (14) has a portion molded to form a hole there through designed to accept and hold in place by a fluid tight seal at least one Hydro-block Air Intake Vent (4). When a user expels urine it is collected by gravity in the Micro Collection Chamber (14) and exits by gravity through the Urine Passage Channel (13) into the Gravity Draining Drainage Hose (11) which is connected to the Micro Collection Chamber (14) by means of the Connector to Catheter Body Retainer (6) which is attached to a Hydro-block Air Intake Coupler (7) that maintains a fluid tight seal by means of a Interconnector Sealing O-Ring (8) to the Quick Disconnect Coupling (9) that is attached to the Gravity Draining Drainage Hose (11).

Once a user has placed the device over her genitalia and it is attached to the user as heretofore described the user may pass urine into the device. During the process of urination gravity and excretion pressure from the user's bladder and urethra will cause the urine to flow into the Micro Collection Chamber (12) and then be drawn away from the genitalia by gravity through the Urine Passage Channel (13) and away from the user into a user selected collection or disposal means. The urine will not be able to accumulate around the genitalia because the Hydro-block Air Intake Vent (4) and the Hydro-block Air Intake Coupler (7) will provide free air flow into the device thereby causing the urine to completely drain away from the user's genitalia and ultimately discharge through the Gravity Draining Drainage Hose (11).

The Gravity Draining embodiment of the Hydro-Block Air Vent External Female Catheter (2) may be manufactured from any suitably non-allergenic flexible material such as silicone rubber to optimize comfort to the user.

3) The Comfort Ring Embodiment of the Hydro-Block Air Vent External Female Catheter.

The Comfort Ring embodiment of both the Gravity Draining embodiment of the Hydro-Block Air Vent External Female Catheter (2), depicted in FIGS. 2, 3, 5, 6A and 6B, and the Pump Draining embodiment of the Hydro-Block Air Vent External Female Catheter (1), depicted in FIGS. 1, 4, 6A and 6B, are each manufactured and comprised of the following components in their respective functional relationships:

The Comfort Ring embodiment of both the Gravity Draining embodiment of the Hydro-Block Air Vent External Female Catheter (2) depicted in 6A and 6B and the Pump Draining Hydro-Block Air Vent External Female Catheter (1) depicted in 6A and 6B are each manufactured and comprised of the same components respectively as outlined above and depicted in FIGS. 1-5, except that each embodiment has the addition of a Comfort Ring (16). The Comfort Ring (16) can be detachable in either the Gravity Draining embodiment or the Pump Draining embodiment as depicted in FIG. 6B or molded into or otherwise attached as depicted in FIG. 6A. The Comfort Ring (16) can be inflatable using the pump or vacuum means described above in the Pump Draining embodiment filling the Comfort Ring (16) with a gas, liquid, gel or beaded polymer. The Comfort Ring (16) is comprised of a suitably flexible material similar to the flexible components of the rest of the device such that a user's body and genitalia will be cushioned thereby when the device is worn in use. The Comfort Ring (16) that is attached or detachable in either the Gravity Draining embodiment or the Pump Draining embodiment may also be a sealed ring that is non-inflatable by a user and is pre-filled with a gas, liquid, gel or beaded polymer.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. Accordingly, the scope of the invention should be determined not by the embodiments illustrated, but by the claims and their legal equivalents which accompany this application.

Having described my invention, I claim:

1. A hydro-block air vent female external catheter comprised of:
an external catheter capable of covering the female genitalia and containing urine therein;
an external catheter attachment means attached to the external catheter;
a liquid blocking air vent attached to the external catheter wherein the liquid blocking air vent permits ambient air to enter into or escape from the external catheter and prevents liquid from escaping from within the external catheter; and
a collection means attached to the external catheter wherein the collection means facilitates the passage of urine from the external catheter to a user selected disposal means.

2. The hydro-block air vent female external catheter of claim 1 wherein the collection means is comprised of:
a liquid sensor;
a liquid hose;
a sensor hose;
a pump and sensor connector wherein the pump and sensor connector is capable of being connected to a user selected pump; and
a pressure relief balloon capable of regulating liquids in the liquid hose such that the liquid sensor will detect the presence of liquids without air bubble interference, wherein the collection means is attached to the external catheter by the external catheter attachment means thereby permitting a user's urine to: pass out of the external catheter; be detected by the liquid sensor thereby electronically transmitting data regarding the presence of the user's urine to the pump and sensor connector through the sensor hose thereby facilitating the activation of the user selected pump which when attached and activated draws urine away from the external catheter exiting the external catheter into the liquid hose, then through the pump and sensor connector and then through the user selected pump to a user selected disposal means.

3. The hydro-block air vent female external catheter of claim 1 wherein the collection means is comprised of:
a drain tube attached to the external catheter; and
a storage bag attached to the drain tube wherein urine being discharged by a user will pass out of the external catheter into the drain tube and then pass into the storage bag.

4. The hydro-block air vent female external catheter of claim 3 wherein the drain tube is further comprised of:
a liquid blocking air vent attached to the drain tube wherein the liquid blocking air vent permits ambient air to enter into or escape from the drain tube and prevents liquid from escaping from within the drain tube.

5. The hydro-block air vent female external catheter of claim 2 wherein the liquid sensor is located in the external catheter such that it will sense the presence of a user's urine upon urination.

6. The hydro-block air vent female external catheter of claim 1 wherein the hydro-block air vent female external catheter is further comprised of:
a flexible tube attached to the external catheter wherein the flexible tube serves to cushion the user's body from the environment while the hydro-block air vent female external catheter is attached to the user for use.

7. The hydro-block air vent female external catheter of claim 6 wherein the flexible tube is filled a substance selected from the group consisting of: a gas, a liquid, a gel or a beaded polymer.

8. The hydro-block air vent female external catheter of claim 6 wherein the flexible tube is removably attached to the external catheter.

9. The hydro-block air vent female external catheter of claim 6 wherein the flexible tube is filled a substance selected from the group consisting of: a gas, a liquid, a gel or a beaded polymer by means of a pump.

10. A method of using the hydro-block air vent female external catheter of claim 1 consisting of the steps of:
selecting the external catheter;
attaching the external catheter on the user's body for use by the attachment means;
positioning the collection means to facilitate a flow of urine from the external catheter to the disposal means.

11. A method of using the hydro-block air vent female external catheter of claim 2 consisting of the steps of:
selecting the external catheter;
attaching the external catheter on the user's body for use by the attachment means;
positioning the collection means to facilitate a flow of urine from the external catheter to the disposal means;
selecting a pump;
connecting the pump to the pump and sensor connector; and
turning the pump on such that when the liquid sensor detects the presence of the user's urine the pump will turn on and draw the urine away from the condom through the liquid hose then through the pump to be discharged in the user selected disposal means.
